(19) United States
(12) Patent Application Publication
(10) Pub. No.: US 2023/0149405 A1
(43) Pub. Date: May 18, 2023

(54) TOPICAL USE OF ERLOTINIB FOR TREATING KERATODERMAS IN CHILDREN

(71) Applicants: LABORATOIRES C.T.R.S., Boulogne-Billancourt (FR); INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR); UNIVERSITE PARIS-SACLAY, Gif-sur-Yvette (FR)

(72) Inventors: Christine BODEMER, Paris (FR); Céline GRECO, Villejuif (FR); Claude BOUCHEIX, Villejuif (FR); Jean-Pascal CONDUZORGUES, Montpellier (FR); Joel SCHLATTER, Paris (FR); Salvatore CISTERNINO, Paris (FR)

(73) Assignees: LABORATOIRES C.T.R.S., Boulogne-Billancourt (FR); INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR); UNIVERSITE PARIS-SACLAY, Gif-sur-Yvette (FR)

(21) Appl. No.: 17/916,904

(22) PCT Filed: Apr. 7, 2021
(86) PCT No.: PCT/EP2021/059008

§ 371 Date: Oct. 4, 2022

(30) Foreign Application Priority Data
Apr. 7, 2020 (WO) 20315126.1

(51) Int. Cl. A61K 31/517; A61K 9/00; A61K 9/107; A61K 9/08; A61P 17/02; A61K 47/10; A61K 47/40; A61K 47/32; A61K 47/38

(52) U.S. Cl. CPC A61K 31/517; A61K 9/0014; A61K 9/107; A61K 9/08; A61P 17/02; A61K 47/10; A61K 47/40; A61K 47/32; A61K 47/38

(57) ABSTRACT

A topical composition including erlotinib and a pharmaceutically acceptable excipient for use in the treatment of a keratoderma in a child, preferably a palmoplantar keratoderma (PPK), wherein the composition is topically administered. The composition may further include a penetration enhancer. The treated subject may be younger than three years old. Also, woven or non-woven fabric support including erlotinib and to dressings, patches, gloves and socks having the support useful in the treatment of a PPK.

### FIELD OF INVENTION

The present invention relates to topical compositions comprising erlotinib for use in the treatment of keratodermas in children, especially palmoplantar keratodermas (PPK).

### BACKGROUND OF INVENTION

Palmoplantar keratodermas (PPK) are a general class of diseases characterized by marked thickening of the skin. PPK may be acquired (e.g., paraneoplastic) or inherited.

Olmsted syndrome (OS) is a keratoderma characterized by the combination of periorificial, keratotic plaques and bilateral palmoplantar keratoderma. OS is a rare condition (prevalence is lower than 1/1000000). OS generally affect male patients, although female cases have been reported. Symptoms typically appear at birth or in early childhood and may be associated with severe pain. Existing treatments only treat the symptoms and temporary reduction of pain. State-of-the art treatments include administration of corticosteroids, emollients, keratolytic agent and retinoids.

Pachyonychia congenita (PC) is an inherited genetic skin condition characterized by palmoplantar keratoderma accompanied with pain, thickened nails, presence of cysts and whitening of mucosa in the mouth. PC is a very rare condition as only 1000 worldwide cases have been reported so far. Although it may appear during later childhood, most of the time PC symptoms begin in the first years of life. Typically, a keratoderma develops on the feet or hands of the subject along with underlying blistering. The symptoms of PC may be either localized or diffuse. Sometimes, a follicular keratosis is observed on the trunk and extremities due to friction points such as the knee, the elbow or the waist. In any case, PC cause severe pain to the subject and may also be responsible for feeding difficulties or walking disabilities.

Erlotinib is an Epidermal Growth Factor Receptor Tyrosine Kinase Inhibitor (EGFR-TKI) that was mainly known as an anticancer agent. Common side-effects of systemic administration of erlotinib are rash (in the majority of patients), diarrhea, loss of appetite, fatigue and partial hair loss.

Treatment of keratodermas by means of oral administration of erlotinib are taught for example in GRECO, C. et al. (JAMA DERMATOLOGY, Vol. 156, No. 2, February 2020, p. 191), ZHANG, A. et al. (JAMA DERMATOLOGY, Vol. 156, No. 2, February 2020, p. 196), KENNER-BELL BRANDI, M. et al. (JOURNAL OF THE AMERICAN ACADEMY OF DERMATOLOGY, Vol. 63, No. 2, August 2010, pp. e58-e59), patent application WO 2009/091889 A1 (UNIV. GEORGETOWN et al.) and THOMAS, B. et al., ACTA DERMATO VENEREOLOGICA, Vol. 100, No. 7, March 2020, pp. 168-176; these documents being hereby incorporated by reference.

Patent application PCT/EP2019/076803 (WO 2020/070239 A1), hereby incorporated by reference, discloses methods of treatment of keratodermas, in particular of Olmsted syndrome, wherein erlotinib is used as an active ingredient for treating the keratoderma. However, in PCT/EP2019/076803 the patients are treated orally (per os). Systemic administration of erlotinib could be unsuitable for children considering the side-effects listed hereinabove.

Therefore, there is an urgent need for new therapeutic strategy for treating PPK such as Olmsted syndrome or pachyonychias. The Applicants surprisingly found that topical administration of erlotinib may provide therapeutic activity while limiting the inconveniences of existing PPK treatments.

### SUMMARY

This invention relates to a topical composition for use in the treatment of a keratoderma; wherein the composition comprises: erlotinib in an amount ranging from about 0.01 to about 10% by weight of the total weight of the composition, and a pharmaceutically acceptable excipient; wherein the composition is topically administered to a subject; and wherein the subject is a human younger than fifteen years old.

According to one embodiment, the keratoderma is a palmoplantar keratoderma, preferably an inherited palmoplantar keratoderma. In one embodiment, the palmoplantar keratoderma is selected from: inherited diffuse palmoplantar keratoderma such as Erythrokeratodermia variabilis, palmoplantar keratoderma of Sybert, Olmsted syndrome or Naegeli Franceschetti Jadassohn syndrome; and inherited focal palmoplantar keratoderma such as Papillon-Lefevre syndrome, Pachyonychia congenita type I, Pachyonychia congenita type II, focal palmoplantar keratoderma with oral mucosal hyperkeratosis or Camisa disease. In one specific embodiment, the palmoplantar keratoderma is selected from Olmsted syndrome, Pachyonychia congenita type I and Pachyonychia congenita type II.

According to one embodiment, the composition comprises erlotinib in an amount ranging from about 0.1 to about 5% by weight of the total weight of the composition. According to one embodiment, the pharmaceutically acceptable excipient comprises a penetration enhancer selected from an alcohol, a polyoxyethylene sorbitan monooleate, a polyoxyethylene (C₁₀-C₁₄) alkyl ether, a propylene glycol (C₆-C₁₀) alkyl ester, a polyethylene glycol (C₆-C₁₀) alkyl ester, a polyethylene glycol, a cyclodextrin and a mixture thereof; preferably the penetration enhancer is selected from ethanol, isopropanol, 2-(2-ethoxyethoxy)ethanol, polysorbate 20, polysorbate 80, polyoxyethylene (4) lauryl ether, propylene glycol monocaprylate, polyethylene glycol caprylic ester, PEG 400, β-cyclodextrin and a mixture thereof. According to one embodiment, the composition is a cream, a liniment, a gel, a lotion, an ointment, a foam, a solution, a suspension, an emulsion, a paste, an aerosolized mixture or a powder.

According to one embodiment, the composition is administered topically to a lesion of the skin. According to one embodiment, the composition is administered topically to a hand and/or a foot. According to one embodiment, the use comprises oral administration of erlotinib to the subject. According to one embodiment, the use comprises oral administration of erlotinib to the subject before and/or after topical administration of the composition to the subject.

According to one embodiment, the subject is a human younger than thirteen years old, preferably younger than ten years old, more preferably younger than seven years old, furthermore preferably younger than five years old.

This invention also relates to a woven or non-woven fabric support comprising erlotinib in an amount ranging from about 0.001 to about 2%, preferably ranging from about 0.01 to about 0.1%, by weight of the total weight of the support. This invention also relates to a dressing or patch comprising the support. This invention relates to a glove or sock comprising the support.

### Definitions

In the present invention, the following terms have the following meanings:
“About” is used herein to mean approximately, roughly, around, or in the region of. When the term “about” is preceding a figure, it means plus or less 10% of the value of said figure. When the term “about” is used in conjunction with a numerical range, it modifies that range by extending the boundaries above and below the numerical values set forth by 10%.
“Active ingredient” and “active pharmaceutical ingredient” and “therapeutic agent” refer to a compound for therapeutic use and relating to health. Especially, an active ingredient may be indicated for treating a disease. An active ingredient may also be indicated for improving the therapeutic activity of another active ingredient for treating a disease.
“Administration”, or a variant thereof (e.g., “administering”), means providing a substance, such as an active ingredient (e.g., erlotinib), alone or as part of a pharmaceutically acceptable composition, to a subject in whom/which a condition, symptom, or disease is to be treated.
“Child” refers to a human younger than fifteen years old, unless otherwise stated.
“Comprise” or a variant thereof (e.g., “comprises”, “comprising”) is used herein according to common patent application drafting terminology. Hence, “comprise” preceded by an object and followed by a constituent means that the presence of a constituent in the object is required (typically as a component of a composition), but without excluding the presence of any further constituent(s) in the object. Moreover, any occurrence of “comprise” or a variant thereof herein also encompasses narrower expression “substantially consist”, further narrower expression “consist of” and any variants thereof (e.g., “consists”, “consisting”), unless otherwise stated.
“Dermatological condition”, “cutaneous condition” and “skin condition” are synonyms and refer to any medical condition that affects the integumentary system including skin, hair, nails and related muscle and/or glands. Typically, the affected part of the integumentary system is skin.
“Dermatological disease”, “cutaneous disease” and “skin disease” are synonyms and refer to a condition of the integumentary system, preferably a skin condition, associated with specific symptoms and signs.
“Human” refers to a subject of both genders and at any stage of development (i.e., neonate, infant, juvenile, adolescent, adult). Preferably, a human is younger than 15 years old, more preferably younger than 3 years old.
“Keratoderma” has its general meaning in the art and refers to a marked thickening of the skin. Keratodermas may be inherited or not. Diffuse keratodermas mainly affect the palms and soles. Focal keratodermas mainly affect pressure areas. Punctate-type keratodermas result in tiny bumps on the palms and soles. Most often the affected skin involves only the palms and soles but it may extend on to the top of the hands and feet as well.
“Palmoplantar keratoderma” or “PPK” refers to any form of persistent thickening of the epidermis of palms and soles and includes genetic as well as acquired conditions. PPK may be acquired in inflammatory skin diseases such as eczema, psoriasis, and lichen planus. PPK has been reported as a paraneoplastic phenomenon.
“Part” or “in parts” are used herein to describe relative amounts of a substance expressed in volume (v/v).
“Penetration enhancer” refers to an agent that improves the transport of molecules such as an active agent (e.g., a drug) into or through the skin. Thus, a penetration enhancer may be used to assist in the delivery of an active ingredient directly to the skin or underlying tissue or indirectly to the site of the disease or a symptom thereof through systemic distribution. A penetration enhancer may be a single substance or a mixture of different substances.
“Pharmaceutically acceptable” means that the ingredients of a pharmaceutical composition are compatible with each other and not deleterious to the patient thereof. European Pharmacopoeia and United States Pharmacopoeia are well-known references in the pharmaceutical field.
“Pharmaceutical acceptable excipient” refers to a substance, such as a carrier, solvent or diluent, in which an active ingredient is formulated and/or administered.
“Pre-walk” refer to a human subject that has never developed the ability to walk consistently in everyday life, i.e., that has not learned to walk. Thus, a pre-walk subject is typically a young human child. Human children typically start to walk between about 10 and about 18 months although significant variations may occur in individual situations. A subject that cannot walk due to a disease or disability is not considered “pre-walk” in the meaning of this definition, because they know (learned) how to walk, despite not being able to. A person skilled in the art (for example a pediatrician) is able to determine without undue burden by routine experiments whether a specific subject qualifies as “pre-walk” or “post-walk” in accordance to medical procedures known in the art (for example pediatric procedures). The opposite of “pre-walk” is “post-walk” which refer to a human subject that has learned to walk, such as for example a normal adult human.
“Solvate” refers to a molecular complex comprising a compound and containing stoichiometric or sub-stoichiometric amounts of one or more pharmaceutically acceptable solvent molecule such as ethanol. The term “hydrate” refers to when said solvent is water.
“Subject” refers to a warm-blooded animal, preferably a mammal, more preferably a human. Preferably, the subject is a “patient”, i.e., a subject who is awaiting the receipt of, or who is receiving medical care, or who is/will be the object of a medical procedure.
“Therapeutically effective amount” (or more simply an “effective amount”) refers to the amount of active agent or active ingredient that is sufficient to achieve the desired therapeutic or prophylactic effect in the patient to which/whom it is administered.
“Topical administration” or “topically administered” refer to the administration of a substance, such as an active ingredient, to the skin or a localized region of the body of a subject. Topical administration can be used for delivering the substance to external body tissues and/or for transdermal administration of the substance.
“Topical formulation” or “topical composition” refer to a composition that may be applied to the skin of a subject. Topical formulations can be used for both topical and transdermal administration of a substance, such as an active ingredient. Preferably, topical formulations are pharmaceutically acceptable.
“Transdermal administration” refers to the administration of a substance, such as an active ingredient, through the skin of a subject. In the invention, transdermal administration is preferably used for delivering the substance to tissues underlying the skin with minimal systemic absorption, however transdermal administration may also generally be used for systemic delivery of the active ingredient.
“Treating” or “treatment” or “alleviation” refers to both therapeutic treatment and prophylactic or preventative measures; wherein the object is to prevent or slow down (lessen) the targeted disease, disorder or condition in a subject in need thereof. Those in need of treatment include those already with the disease, disorder or condition as well as those prone to have the disorder or those in whom the disorder is to be prevented. A subject is successfully “treated” for a disease, disorder or condition if, after receiving a therapeutic amount of an substance or composition according to the present invention, the subject shows observable and/or measurable reduction in or absence of one or more of the following: reduction in the number of pathogenic cells; reduction in the percent of total cells that are pathogenic; relief to some extent, of one or more of the symptoms associated with the specific disease, disorder or condition; reduced morbidity and mortality; and/or improvement in quality of life issues. The above parameters for assessing successful treatment and improvement in the disease, disorder or condition are readily measurable by routine procedures familiar to a physician.

### DETAILED DESCRIPTION

This invention relates to a topical composition comprising erlotinib for use in the topical treatment of a dermatological disease or a dermatological condition.

“Erlotinib” is (6,7-bis-(2-methoxyethoxy)-4-quinazolin-4-yl]-(3-ethynylphenyl)amine), CAS number [183321-74-6]. Erlotinib has the structure of formula:

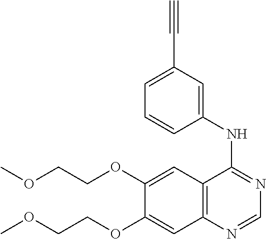 [SMILES: C#Cc1cccc(Nc2ncnc3cc(OCCOC)c(OCCOC)cc23)c1]

All references to erlotinib include references to salts, solvates, multi-component complexes and liquid crystals thereof. All references to erlotinib include references to polymorphs and crystal habits thereof.

All references to erlotinib include references to all possible stereoisomers and includes not only the racemic compounds but the individual enantiomers and their non-racemic mixtures as well. When a compound is desired as a single enantiomer, such single enantiomer may be obtained by stereospecific synthesis, by resolution of the final product or any convenient intermediate, or by chiral chromatographic methods as each are known in the art. Resolution of the final product, an intermediate, or a starting material may be carried out by any suitable method known in the art.

All references to erlotinib include references to all possible pharmaceutically acceptable salts. Pharmaceutically acceptable salts of erlotinib include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts. Hemisalts of acids and bases may also be formed, for example, hemisulphate and hemicalcium salts. Erlotinib may contain an acidic group as well as a basic group and may thus form internal salts, and such compounds are within the scope of the invention. Erlotinib may contain a hydrogen-donating heteroatom, and the invention also covers salts and/or isomers formed by transfer of said hydrogen atom to a basic group or atom within the molecule. Pharmaceutically acceptable salts of erlotinib may be prepared by one or more of these methods: (i) by reacting erlotinib with the desired acid; (ii) by reacting erlotinib) with the desired base; (iii) by removing an acid- or base-labile protecting group from a suitable precursor of erlotinib or by ring-opening a suitable cyclic precursor, e.g., a lactone or lactam, using the desired acid; and/or (iv) by converting one salt of erlotinib to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to almost non-ionized.

The composition comprises erlotinib in an amount ranging from 0.001 to 20% by weight of the total weight of the composition. According to one embodiment, the composition comprises erlotinib in an amount ranging from about 0.001 to about 20%, preferably from about 0.002 to about 16%, more preferably from about 0.003 to about 12%, furthermore preferably from about 0.004 to about 8%, furthermore preferably from about 0.005 to about 4%, by weight of the total weight of the composition. According to one preferred embodiment, the composition comprises erlotinib in an amount ranging from 0.01 to 10% by weight of the total weight of the composition. According to one preferred embodiment, the composition comprises erlotinib in an amount ranging from about 0.01 to about 10%, preferably from about 0.02 to about 8%, more preferably from about 0.03 to about 6%, furthermore preferably from about 0.04 to about 4%, furthermore preferably from about 0.05 to about 2%, by weight of the total weight of the composition. In one embodiment, the composition comprises erlotinib in an amount ranging from 0.1 to 5% by weight of the total weight of the composition. In one embodiment, the composition comprises erlotinib in an amount ranging from about 0.1 to about 5%, preferably from about 0.2 to about 4%, more preferably from about 0.3 to about 3%, furthermore preferably from about 0.4 to about 2%, furthermore preferably from about 0.5 to about 1%, by weight of the total weight of the composition. In one specific embodiment, the composition comprises erlotinib in an amount ranging from 0.1 to 4.5%, from 0.25 to 4%, from 0.5 to 3%, from 0.75 to 3.5%, from 1 to 3%, from 1.25 to 2.5% or from 1.5 to 2%, by weight of the total weight of the composition. In one specific embodiment, the composition comprises erlotinib in an amount ranging from about 0.1 to about 4.5%, from about 0.25 to about 4%, from about 0.5 to about 3%, from about 0.75 to about 3.5%, from about 1 to about 3%, from about 1.25 to about 2.5% or from about 1.5 to about 2%, by weight of the total weight of the composition. All ranges recited in this paragraph by weight of the total weight of the composition (w/w) are herein further recited (i) by weight of the total volume of the composition (w/v) or (ii) by volume of the total volume of the composition (v/v).

The composition comprises a pharmaceutically acceptable excipient. The suitable amount of a pharmaceutically acceptable excipient in the composition may be determined and/or adjusted by a person skilled in the art. According to one embodiment, the excipient comprises erlotinib. Pharmaceutically acceptable excipients for preparing the compositions of the invention may for instance be selected from antioxidants, binders, buffers and pH adjusters, chelating agents, colorants, diluents and fillers (including thickening agents), emollients, emulsifiers, glidants and antiadherents, humectants, lubricants, plasticizers, preservatives (including antimicrobials), propellants, protective colloids, solvents (including cosolvents), surfactants (including cosurfactants), suspending agents, viscosifiers (viscosity modulator agents) and mixtures thereof. These and others pharmaceutically acceptable excipients are disclosed in “Remington: Essentials of pharmaceutics” (Edited by Linda Felton, Pharmaceutical Press 2013, London,), the content of which is hereby incorporated by reference.

According to one embodiment, the composition comprises at least one antioxidant. In one embodiment, the composition comprises at least one antioxidant selected from alpha-tocopherol (vitamin E), ascorbic acid (vitamin C), butylated hydroxyanisole (BHA), butylated hydroxytoluene (BHT), citric acid, sodium bisulfite, sodium metabisulfite and a mixture thereof. According to one embodiment, the composition comprises at least one binder. In one embodiment, the composition comprises at least one binder selected from alginic acid, sodium alginate, carboxymethyl cellulose sodium (CMC), microcrystalline cellulose (MCC), powdered cellulose, confectioner's sugar, dextrin, dextrose, ethylcellulose, guar gum, hydroxypropyl cellulose (HPC), hypromellose (HPMC), lactose, maltodextrin, methylcellulose, povidone, starch, tragacanth, zein and a mixture thereof. According to one embodiment, the composition comprises at least one buffer or pH-adjusting agent. In one embodiment, the composition comprises at least one buffer or pH-adjusting agent selected from acetic acid/acetate, boric acid/borate (borax), carbonate, citric acid/citrate, gluconate, histidine, hydrochloric acid, lactate, potassium hydroxide, phosphoric acid/sodium phosphate or disodium phosphate or tri-sodium phosphate, sodium carbonate, sodium hydrogen carbonate, sodium hydroxide, tris-base buffer, tromethamine and a mixture thereof. According to one embodiment, the composition comprises at least one diluent or filler. In one embodiment, the composition comprises at least one diluent or filler selected from calcium carbonate, calcium sulphate, microcrystalline cellulose (MCC), powdered cellulose, dextrates, dextrin, dextrose, kaolin, lactose, maltodextrin, mannitol, starch, sucrose and a mixture thereof. According to one embodiment, the composition comprises at least one emollient. In one embodiment, the composition comprises at least one emollient selected from glycerin (glycerol), glyceryl monostearate, isopropyl myristate, petrolatum, polyethylene glycols and a mixture thereof. According to one embodiment, the composition comprises at least one emulsifier. In one embodiment, the composition comprises at least one emulsifier selected from carbomer, carrageenan, lanolin, lecithin, mineral oil, oleic acid, oleyl alcohol, pectin, poloxamer, polyoxyethylene sorbitan fatty acid esters, sorbitan esters, triethanolamine and a mixture thereof. According to one embodiment, the composition comprises at least one glidant or antiadherent. In one embodiment, the composition comprises at least one glidant or antiadherent selected from colloidal silicon dioxide, talc and a mixture thereof. According to one embodiment, the composition comprises at least one humectant. In one embodiment, the composition comprises at least one humectant selected from glycerin, propylene glycol, sorbitol, triethanolamine and a mixture thereof. According to one embodiment, the composition comprises at least one lubricant. In one embodiment, the composition comprises at least one lubricant selected from calcium stearate, glyceryl monostearate, isopropyl myristate, magnesium stearate, polyvinyl alcohol, sodium stearyl fumarate, stearic acid, talc and a mixture thereof. According to one embodiment, the composition comprises at least one plasticizer. In one embodiment, the composition comprises at least one plasticizer selected from glycerin, propylene glycol, triacetin, triethanolamine and a mixture thereof. According to one embodiment, the composition is free of preservative agent, i.e., the composition is “preservative-free”. According to another embodiment, the composition comprises at least one preservative agent. In one embodiment, the composition comprises at least one preservative agent selected from benzalkonium chloride (BAK), boric acid, butylated hydroxyanisole (BHA), butylated hydroxytoluene (BHT), butylparaben, ethanol, methylparaben, phenol, phenethyl alcohol, potassium sorbate, propylene glycol, propylparaben, sorbic acid and a mixture thereof. According to one embodiment, the composition comprises at least one propellant. In one embodiment, the composition comprises at least one propellant selected from difluoroethane, nitrogen and a mixture thereof. According to one embodiment, the composition comprises at least one protective colloid. In one embodiment, the composition comprises at least one protective colloid selected from hydroxypropyl cellulose (HPC), hypromellose (HPMC), methylcellulose and a mixture thereof. According to one embodiment, the composition comprises at least one solvent. In one embodiment, the composition comprises at least one solvent selected from water-based carrier, organic solvent, oil and a mixture thereof. In one specific embodiment, the water-based carrier is selected from water, buffered water, Ringer's solution, saline, sugar solution, hydroalcoholic solution and a mixture thereof. In one specific embodiment, the organic solvent is an alcohol, preferably selected from ethanol, isopropanol, glycerin (glycerol), 2-propanol, propylene glycol, 2-(2-ethoxyethoxy)ethanol and a mixture thereof. In one specific embodiment, the oil is selected from fatty acid, mineral oil (such as petrolatum, liquid paraffin, heavy mineral oil or light mineral oil), triglycerides oil, vegetable oil (such as castor oil, corn oil, olive oil, soybean oil, sesame oil, cotton seed oil or sweet almond oil) and a mixture thereof. According to one embodiment, the composition comprises at least one surfactant. In one embodiment, the composition comprises at least one surfactant selected from polyethylene glycols, polyoxyethylene sorbitan fatty acid esters, sodium lauryl sulfate, sorbitan esters and a mixture thereof. According to one embodiment, the composition comprises at least one suspending agent. In one embodiment, the composition comprises at least one suspending agent selected from acacia, agar, carbomer, carboxymethyl cellulose sodium (CMC), carrageenan, microcrystalline cellulose and sodium carboxymethyl, cellulose co-processed, colloidal silicon dioxide, dextrin, guar gum, hydroxypropyl cellulose (HPC), hypromellose (HPMC), kaolin, methylcellulose, pectin, polyvinyl alcohol, povidone, tragacanth and a mixture thereof. According to one embodiment, the composition comprises at least one viscosifier. In one embodiment, the composition comprises at least one viscosifier selected from acacia, agar, sodium alginate, bentonite, carbomer, carboxymethyl cellulose sodium (CMC), guar gum, hydroxypropyl cellulose (HPC), hypromellose (HPMC), methylcellulose, pectin and a mixture thereof.

According to one embodiment, the composition comprises the excipient(s) in a total amount ranging from 0.001 to 99.999% by weight of the total weight of the composition. In one embodiment, the composition comprises the excipient(s) in a total amount ranging from 0.01 to 99.999% by weight of the total weight of the composition. In one specific embodiment, the composition comprises the excipient(s) in a total amount ranging from 0.1 to 99.99% by weight of the total weight of the composition. In one further specific embodiment, the composition comprises the excipient(s) in a total amount ranging from 1 to 99.9% by weight of the total weight of the composition. In one further specific embodiment, the composition comprises the excipient(s) in a total amount ranging from about 1 to about 99.9%, preferably from about 2 to about 90%, more preferably from about 5 to about 80%, furthermore preferably from about 10 to about 70%, furthermore preferably from about 20 to about 60%, furthermore preferably from about 30 to about 50%, by weight of the total weight of the composition. All ranges recited in this paragraph by weight of the total weight of the composition (w/w) are herein further recited (i) by weight of the total volume of the composition (w/v) or (ii) by volume of the total volume of the composition (v/v).

According to one preferred embodiment, the excipient comprises a penetration enhancer. In the invention, the penetration enhancer aims at improving the transport of erlotinib into or through the skin, where erlotinib is topically administrated. Penetration enhancers may be solvents, surfactants or miscellaneous substances susceptible to improve transdermal penetration of erlotinib such as complexing agents.

In one embodiment, the penetration enhancer is selected from:
water, alcohols (such as methanol, ethanol, 2-propanol or 2-(2-ethoxyethoxy)ethanol), alkyl methyl sulfoxides (such as dimethyl sulfoxide, decylmethyl sulfoxide or tetradecylmethyl sulfoxide), pyrrolidones (2-pyrrolidone, N-Methyl-2-pyrrolidone, N-(2-hydroxyethyl)pyrrolidone), laurocapram, acetone, dimethyl acetamide, dimethyl formamides, tetrahydrofurfuryl alcohol;
1-α-amino acids, anionic surfactants, cationic surfactants, amphoteric surfactants, non-ionic surfactants, fatty acids, fatty alcohols;
clofibric acid amides, hexamethylene lauramide, proteolytic enzymes, terpenes and sesquiterpenes (such as α-bisabolol and d-limonene), urea, N,N-ethyl-m-toluamide; and
a mixture thereof.

In one embodiment, the penetration enhancer is a solvent. A suitable solvent for solubilizing erlotinib may be selected from alcohols such as ethanol, isopropanol, 2-(2-ethoxyethoxy)ethanol (commercial brand names such as Transcutol®) and a mixture thereof. In one specific embodiment, the penetration enhancer is 2-(2-ethoxyethoxy)ethanol.

In one embodiment, the penetration enhancer is a surfactant. In one specific embodiment, the surfactant is selected from:
a polyoxyethylene sorbitan monooleate such as polyoxyethylene (20) sorbitan monooleate (“polysorbate 20”, commercial brand names such as Alkest TW 20, Scattics or Tween 20) or polyoxyethylene (80) sorbitan monooleate (“polysorbate 80”, commercial brand names such as Alkest TW 80, Scattics or Tween 80);
a polyoxyethylene (C₁₀-C₁₄) alkyl ether such as polyoxyethylene (4) lauryl ether (commercial brand names such as Brij® 30);
a propylene glycol (C₆-C₁₀) alkyl ester such as propylene glycol monocaprylate (“PGMC”, commercial brand names such as Capryol 90);
a polyethylene glycol (C₆-C₁₀) alkyl ester such as polyethylene glycol caprylic ester (commercial brand names such as Labrasol);
a polyethylene glycol such as polyethylene glycol 400 (“PEG 400”, commercial brand names such as Carbowax or Macrogol) and
a mixture thereof.

In one embodiment, the penetration enhancer is a complexing agent. A complexing agent forms a complex with erlotinib and can be used to transport it into deeper tissues. In one specific embodiment, the complexing agent is a cyclodextrin such as (alpha)-cyclodextrin, (beta)-cyclodextrin or (gamma)-cyclodextrin. In one further specific embodiment, the cyclodextrin is (beta)-cyclodextrin.

In one embodiment, the composition comprises the penetration enhancer(s) in a total amount ranging from 0.01 to 99.999% by weight of the total weight of the composition. In one embodiment, the composition comprises the penetration enhancer(s) in a total amount ranging from about 0.01 to about 99.9%, preferably from about 0.02 to about 80%, more preferably from about 0.05 to about 60%, furthermore preferably from about 0.1 to about 40%, furthermore preferably from about 0.2 to about 20%, furthermore preferably from about 0.3 to about 1%, by weight of the total weight of the composition. In one specific embodiment, the composition comprises the penetration enhancer(s) in a total amount ranging from 0.1 to 99.99% by weight of the total weight of the composition.

In one specific embodiment, the composition comprises the penetration enhancer(s) in a total amount ranging from about 0.1 to about 99.9%, preferably from about 0.2 to about 90%, more preferably from about 0.5 to about 80%, furthermore preferably from about 1 to about 70%, furthermore preferably from about 2 to about 60%, furthermore preferably from about 3 to about 50%, by weight of the total weight of the composition. In one further specific embodiment, the composition comprises the penetration enhancer(s) in a total amount ranging from 1 to 99.9% by weight of the total weight of the composition. In one further specific embodiment, the composition comprises the penetration enhancer(s) in a total amount ranging from about 1 to about 99.9%, preferably from about 2 to about 80%, more preferably from about 5 to about 70%, furthermore preferably from about 10 to about 60%, furthermore preferably from about 20 to about 50%, furthermore preferably from about 30 to about 40%, by weight of the total weight of the composition. All ranges recited in this paragraph by weight of the total weight of the composition (w/w) are herein further recited (i) by weight of the total volume of the composition (w/v) or (ii) by volume of the total volume of the composition (v/v).

In one further specific embodiment, the composition comprises the penetration enhancer(s) in a total amount ranging from 0.1 to 20%, preferably from 1 to 10%, by weight of the total weight of the composition. In one further specific embodiment, the composition comprises the penetration enhancer(s) in a total amount ranging from about 0.1 to about 20%, preferably from about 1 to about 10%, by weight of the total weight of the composition. In another further specific embodiment, the composition comprises the penetration enhancer(s) in a total amount ranging from 0.5 to 20%, preferably from 5 to 10%, by weight of the total weight of the composition. In another further specific embodiment, the composition comprises the penetration enhancer(s) in a total amount ranging from about 0.5 to about 20%, preferably from about 5 to about 10%, by weight of the total weight of the composition. In another further specific embodiment, the composition comprises the penetration enhancer(s) in a total amount ranging from 1 to 65%, preferably from 10 to 45%, by weight of the total weight of the composition. In another further specific embodiment, the composition comprises the penetration enhancer(s) in a total amount ranging from about 1 to about 65%, preferably from about 10 to about 45%, by weight of the total weight of the composition. In another further specific embodiment, the composition comprises the penetration enhancer(s) in a total amount ranging from 2 to 60%, preferably from 20 to 40%, by weight of the total weight of the composition. In another further specific embodiment, the composition comprises the penetration enhancer(s) in a total amount ranging from about 2 to about 60%, preferably from about 20 to about 40%, by weight of the total weight of the composition. In another further specific embodiment, the composition comprises the penetration enhancer(s) in a total amount ranging from 2 to 70%, preferably from 10 to 50%, by weight of the total weight of the composition. In another further specific embodiment, the composition comprises the penetration enhancer(s) in a total amount ranging from about 2 to about 70%, preferably from about 10 to about 50%, by weight of the total weight of the composition. All ranges recited in this paragraph by weight of the total weight of the composition (w/w) are herein further recited (i) by weight of the total volume of the composition (w/v) or (ii) by volume of the total volume of the composition (v/v).

One advantage of the compositions of the inventions is that erlotinib is properly solubilized therein. Another advantage of the compositions of the inventions is that appropriate transdermal delivery of erlotinib is achieved, which leads to potent topical treatment of keratodermas. When treating keratodermas, it may not be desired that erlotinib is found in others part of the body except the skin, because of the side-effects of systemic administration of erlotinib. Another advantage of the compositions of the inventions is that topical treatment of keratodermas is achieved without systemic delivery of erlotinib, thereby minimizing unwanted side-effects.

According to a first embodiment, the composition comprises erlotinib as sole active ingredient. According to a second embodiment, the composition further comprises at least another active ingredient. Suitable further active ingredients include antiallergenic agents, anti-inflammatory agents, antineoplastic agents (such as carmustine, cisplatin, mitomycin or fluorouracil), immunological drugs (such as vaccines or immune stimulants), anti-angiogenic compounds, antibodies or antibodies fragments, gene fragments, immunomodulators, secretagogues, antithrombotic and vasodilator agents, antioxidants, antivirals, antibiotics, antifungals, antibacterials and mixtures thereof. In one embodiment, the other active ingredient is urea.

In one embodiment, the composition comprises the other active ingredient(s) in a total amount ranging from 0.1 to 10% by weight of the total weight of the composition. In one embodiment, the composition comprises the other active ingredient(s) in a total amount ranging from about 0.1 to about 10%, preferably from about 0.2 to about 8%, more preferably from about 0.3 to about 6%, furthermore preferably from about 0.4 to about 4%, furthermore preferably from about 0.5 to about 2%, by weight of the total weight of the composition. In one specific embodiment, the composition comprises the other active ingredient(s) in a total amount ranging from 0.1 to 5% by weight of the total weight of the composition. In one embodiment, the composition comprises the other active ingredient(s) in a total amount ranging from about 0.1 to about 5%, preferably from about 0.2 to about 4%, more preferably from about 0.3 to about 3%, furthermore preferably from about 0.4 to about 2%, furthermore preferably from about 0.5 to about 1%, by weight of the total weight of the composition. In one specific embodiment, the composition comprises the other active ingredient(s) in a total amount ranging from 0.1 to 4.5%, 0.25 to 4%, 0.5 to 3%, 0.75 to 3.5%, 1 to 3%, 1.25 to 2.5% or 1.5 to 2%, by weight of the total weight of the composition. In one specific embodiment, the composition comprises the other active ingredient(s) in a total amount ranging from about 0.1 to about 4.5%, about 0.25 to about 4%, about 0.5 to about 3%, about 0.75 to about 3.5%, about 1 to about 3%, about 1.25 to about 2.5% or about 1.5 to about 2%, by weight of the total weight of the composition. All ranges recited in this paragraph by weight of the total weight of the composition (w/w) are herein further recited (i) by weight of the total volume of the composition (w/v) or (ii) by volume of the total volume of the composition (v/v).

The compositions of the invention may conveniently be presented in dosage unit form (e.g., a single dose unit) and may be prepared by any of the methods well known in the art of pharmacy. All methods include the step of bringing the active ingredient into association with the excipient which constitutes one or more accessory ingredients. In general, the compositions are prepared by uniformly and intimately bringing the active ingredient into association with a liquid excipient or a finely divided solid excipient or both, and then, if necessary, shaping the product into the desired formulation. The preparation process is preferably sterile.

According to one embodiment, the composition is a cream, a liniment, a gel, a lotion, an ointment, a foam, a solution, a suspension, an emulsion, a paste, an aerosolized mixture or a powder. According to one embodiment, the composition is a nanoparticulate erlotinib formulation as disclosed in patent EP 1 871 345 B1 (Elan Pharma International Limited County Westmeath), the content of which is hereby incorporated by reference.

According to one embodiment, the composition is a pharmaceutical composition. According to one embodiment, the composition is a medicament.

According to one embodiment, the composition is not in the form of a gel. According to one embodiment, the composition does not comprise dimethyl sulfoxide (DMSO). According to one embodiment, the composition does not comprise ethanol. According to one embodiment, the composition does not comprise isopropyl alcohol. According to one embodiment, the composition does not comprise dimethyl isosorbide. According to one embodiment, the composition does not comprise isopropyl myristate. According to one embodiment, the composition does not comprise oleic acid. According to one embodiment, the composition does not comprise a polyethylene glycol. According to one embodiment, the composition does not comprise hexylene glycol. According to one embodiment, the composition does not comprise glycofurol. According to one embodiment, the composition does not comprise propylene glycol. According to one embodiment, the composition does not comprise glycerin. According to one embodiment, the composition is not in the form of a patch.

In the invention, the composition is for use in a topical treatment, i.e., the composition comprising erlotinib as active ingredient is administrated topically.

According to one preferred embodiment, the composition is for use in the topical treatment of a keratoderma. According to one embodiment, the composition is for use in the topical treatment of an acquired keratoderma. According to one embodiment, the composition is for use in the topical treatment of an inherited keratoderma.

According to one embodiment, the keratoderma is palmoplantar keratoderma (PPK). In one embodiment, the PPK is selected from diffuse palmoplantar keratoderma and focal palmoplantar keratoderma. In one specific embodiment, the PPK is a diffuse palmoplantar keratoderma. In one specific embodiment, the PPK is a focal palmoplantar keratoderma.

According to one embodiment, the PPK is any inherited PPK disclosed in GUERRA, L. et al., Journal of the European Academy of Dermatology and Venereology, May 2018, Vol. 32, No. 5, pp. 704-719 (I. Non-syndromic classification), hereby incorporated by reference.

In one embodiment, the PPK is selected from:
I-1.1 PALMOPLANTAR KERATODERMA WITH SENSORINEURAL DEAFNESS [heterozygous GJB2 mutations cause Vohwinkel syndrome (VS), Bart-Pumphrey syndrome (BPS) and palmoplantar keratoderma with deafness]
   1.1.1 Mutilating palmoplantar keratoderma with deafness due to GJB2 mutations—classic variant (Vohwinkel syndrome),
   1.1.2 Palmoplantar keratoderma with leukonychia, knuckle pads and deafness due to GJB2 mutations (Bart-Pumphrey syndrome),
   1.1.3 Palmoplantar keratoderma with deafness, due to GJB2 mutations,
   1.1.4 Palmoplantar keratoderma with deafness due to MTTS1 gene mutation;
I-1.2 PALMOPLANTAR KERATODERMA WITH PROMINENT MUCOSAL INVOLVEMENT [Haim-Munk (HMS) and the Papillon-Lefevre (PLS) syndromes]
   1.2.1 Palmoplantar keratoderma with periodontitis due to CTSC mutation (Haim-Munk syndrome),
   1.2.2 Palmoplantar keratoderma with periodontitis due to CTSC mutation (Papillon-Lefevre syndrome),
   1.2.3 Focal palmoplantar keratoderma with gingival keratosis,
   1.2.4 Hypotrichosis, osteolysis, periodontitis, palmoplantar keratoderma (HOPP syndrome) Van Steensel syndrome,
   1.2.5 Tylosis with esophageal cancer, due to RHBDF2 (iRHOM2) mutation (Howell-Evans syndrome),
   1.2.6 Multiple self-healing palmoplantar carcinoma due to NLRP1 mutation;
I-1.3 PALMOPLANTAR KERATODERMA WITH CARDIOMYOPATHY AND WOOLLY HAIR
   1.3.1 Palmoplantar keratoderma with arrhythmogenic right ventricular cardiomyopathy and woolly hair due to JUP mutations (Naxos disease),
   1.3.2 Palmoplantar keratoderma with left ventricular cardiomyopathy and woolly hair due to DSP mutations (Carvajal syndrome, autosomal recessive),
   1.3.3 Dilated cardiomyopathy with woolly hair, keratoderma and tooth agenesis due to DSP mutations (Carvajal syndrome, autosomal dominant variant),
   1.3.4 Arrhythmogenic right ventricular dysplasia, with mild palmoplantar keratoderma and woolly hair, due to DSC2 mutations;
I-1.4 PALMOPLANTAR KERATODERMAS WITH OTHER SYSTEMIC SIGNS
   1.4.1 Tyrosinemia type II due to TAT mutations (Oculocutaneous tyrosinemia, keratosis palmoplantaris with corneal dystrophy, Richner-Hanhart syndrome),
   1.4.2 Palmoplantar keratoderma, severe dermatitis, multiple allergies and metabolic wasting (SAM syndrome), due to DSG1 mutations,
   1.4.3 Palmoplantar keratodermas, squamous cell carcinomas and disorders of sex development due to RSPO1 mutations,
   1.4.4 Guttate hypopigmentation and punctate palmoplantar keratoderma with or without ectopic calcification, due to ENPP1 mutations (Cole disease),
   1.4.5 Aquagenic wrinkling of the palms, AWP (aquagenic acrokeratoderma) associated with mutant CFTR alleles; and
I-2. PALMOPLANTAR KERATODERMAS IN OTHER GENETIC DISEASES.

According to one embodiment, the PPK is selected from inherited PPK disclosed in GUERRA, L. et al., Journal of the European Academy of Dermatology and Venereology, June 2018, Vol. 32, No. 6, pp. 899-925 (II. Inherited PPK syndromic classification), hereby incorporated by reference.

In one embodiment, the PPK is selected from:
II-1.1 PALMOPLANTAR KERATODERMA WITH SENSORINEURAL DEAFNESS [heterozygous GJB2 mutations cause Vohwinkel syndrome (VS), Bart-Pumphrey syndrome (BPS) and palmoplantar keratoderma with deafness]
   1.1.1 Mutilating palmoplantar keratoderma with deafness due to GJB2 mutations—classic variant (Vohwinkel syndrome),
   1.1.2 Palmoplantar keratoderma with leukonychia, knuckle pads and deafness due to GJB2 mutations (Bart-Pumphrey syndrome),
   1.1.3 Palmoplantar keratoderma with deafness, due to GJB2 mutations,
   1.1.4 Palmoplantar keratoderma with deafness due to MTTS1 gene mutation;
II-1.2 PALMOPLANTAR KERATODERMA WITH PROMINENT MUCOSAL INVOLVEMENT [Haim-Munk (HMS) and the Papillon-Lefevre (PLS) syndromes]
   1.2.1 Palmoplantar keratoderma with periodontitis due to CTSC mutation (Haim-Munk syndrome),
   1.2.2 Palmoplantar keratoderma with periodontitis due to CTSC mutation (Papillon-Lefevre syndrome),
   1.2.3 Focal palmoplantar keratoderma with gingival keratosis,
   1.2.4 Hypotrichosis, osteolysis, periodontitis, palmoplantar keratoderma (HOPP syndrome) Van Steensel syndrome,
   1.2.5 Tylosis with esophageal cancer, due to RHBDF2 (iRHOM2) mutation (Howell-Evans syndrome),
   1.2.6 Multiple self-healing palmoplantar carcinoma due to NLRP1 mutation;
II-1.3 PALMOPLANTAR KERATODERMA WITH CARDIOMYOPATHY AND WOOLLY HAIR
   1.3.1 Palmoplantar keratoderma with arrhythmogenic right ventricular cardiomyopathy and woolly hair due to JUP mutations (Naxos disease),
   1.3.2 Palmoplantar keratoderma with left ventricular cardiomyopathy and woolly hair due to DSP mutations (Carvajal syndrome, autosomal recessive),
   1.3.3 Dilated cardiomyopathy with woolly hair, keratoderma and tooth agenesis due to DSP mutations (Carvajal syndrome, autosomal dominant variant),
   1.3.4 Arrhythmogenic right ventricular dysplasia, with mild palmoplantar keratoderma and woolly hair, due to DSC2 mutations;
II-1.4 PALMOPLANTAR KERATODERMAS WITH OTHER SYSTEMIC SIGNS
   1.4.1 Tyrosinemia type II due to TAT mutations (Oculocutaneous tyrosinemia, keratosis palmoplantaris with corneal dystrophy, Richner-Hanhart syndrome),
   1.4.2 Palmoplantar keratoderma, severe dermatitis, multiple allergies and metabolic wasting (SAM syndrome), due to DSG1 mutations,
   1.4.3 Palmoplantar keratodermas, squamous cell carcinomas and disorders of sex development due to RSPO1 mutations,
   1.4.4 Guttate hypopigmentation and punctate palmoplantar keratoderma with or without ectopic calcification, due to ENPP1 mutations (Cole disease),
   1.4.5 Aquagenic wrinkling of the palms, AWP(aquagenic acrokeratoderma) associated with mutant CFTR alleles; and
II-2. PALMOPLANTAR KERATODERMAS IN OTHER GENETIC DISEASES.

In one specific embodiment, the diffuse palmoplantar keratoderma is selected from Erythrokeratodermia variabilis, palmoplantar keratoderma of Sybert, Olmsted syndrome and Naegeli-Franceschetti-Jadassohn syndrome. In one further specific embodiment, the diffuse palmoplantar keratoderma is Erythrokeratodermia variabilis. In one further specific embodiment, the diffuse palmoplantar keratoderma is palmoplantar keratoderma of Sybert. In one further specific embodiment, the diffuse palmoplantar keratoderma is Olmsted syndrome. In one further specific embodiment, the diffuse palmoplantar keratoderma is Naegeli-Franceschetti-Jadassohn syndrome.

In one specific embodiment, the focal palmoplantar keratoderma is selected from Papillon-Lefevre syndrome, Pachyonychia congenita type I, Pachyonychia congenita type II, focal palmoplantar keratoderma with oral mucosal hyperkeratosis and Camisa disease. In one further specific embodiment, the focal palmoplantar keratoderma is Papillon-Lefevre syndrome. In one further specific embodiment, the focal palmoplantar keratoderma is selected from Pachyonychia congenita type I and Pachyonychia congenita type II. In one further specific embodiment, the focal palmoplantar keratoderma is Pachyonychia congenita type I. In one further specific embodiment, the focal palmoplantar keratoderma is Pachyonychia congenita type II. In one further specific embodiment, the focal palmoplantar keratoderma is focal palmoplantar keratoderma with oral mucosal hyperkeratosis. In one further specific embodiment, the focal palmoplantar keratoderma is Camisa disease. In one specific embodiment, the keratoderma is selected from Olmsted syndrome, Pachyonychia congenita type I and Pachyonychia congenita type II.

According to one embodiment, the composition is administered topically to a lesion of the skin, e.g., a lesion on the skin of a hand or a foot. According to one embodiment, the composition is administered topically to a hand and/or a foot. In one embodiment, the composition is administered topically to at least one hand of a subject. In one specific embodiment, the composition is administered topically on the palm of at least one hand of a subject. In a first embodiment, the palm includes the palm that is part of the fingers (“administration on the full palm”). In a second embodiment, the palm does not include the palm that is part of the fingers (“administration on the main palm”). In one embodiment, the composition is administered topically to at least one foot of a subject. In one specific embodiment, the composition is administered topically on the sole of at least one foot of a subject. In a first embodiment, the sole includes the sole that is part of the toes (“administration on the full sole”). In a second embodiment, the sole does not include the sole that is part of the toes (“administration on the main sole”).

According to a first embodiment, the composition is applied with a light massage or rubbing of the composion on the skin, in order to improve penetration of the composition. According to a second embodiment, the composition is administrated without any massage nor rubbing of the composion on the skin. According to one embodiment, protecting device such as for example a dressing, a patch or a piece of clothing is applied over the composition after application.

Another advantage of the composition of the invention is that it does not diffuse outside of the area where it is applied, which avoids adverse effects on the skin, especially adverse effects on healthy skin (i.e., skin not affected by a keratoderma).

According to one embodiment, the subject is submitted to oral administration of erlotinib according to methods for treating keratoderma known of the art before the topical use of erlotinib according to the invention as described herein, simultaneously to the topical use of erlotinib according to the invention as described herein and/or after the topical use of erlotinib according to the invention as described herein. In one embodiment, oral administration of erlotinib is made before topical administration of erlotinib (sequential administration). In one embodiment, oral and topical administrations of erlotinib are simultaneous. In one embodiment, oral administration of erlotinib is made after topical administration of erlotinib (sequential administration).

According to one embodiment, the use according to the invention further comprises a step of oral administration of erlotinib according to methods for treating keratoderma known of the art. In one embodiment, the step of oral administration of erlotinib is made before the step of topical administration of erlotinib (sequential administration). In one embodiment, the steps of oral and topical administration of erlotinib are simultaneous. In one embodiment, the step of oral administration of erlotinib is made after the step of topical administration of erlotinib (sequential administration).

In one embodiment, topical administration of erlotinib is used in replacement of oral administration of erlotinib when the intensity of lesions and/or plantar keratoses in the subject reaches a predetermined level, such as for example a reduction of about 30%, about 40%, about 50%, about 60%, about 70%, about 80% or about 90% of symptoms. Intensity of lesions and/or plantar keratoses in a subject can be assessed by a skilled person using methods known in the art.

According to one embodiment, the subject who is treated for the keratoderma is a human younger than fifteen years old. In one embodiment, the subject is a human younger than fourteen years old. In one embodiment, the subject is a human younger than thirteen years old. In one embodiment, the subject is a human younger than twelve years old. In one embodiment, the subject is a human younger than eleven years old. In one embodiment, the subject is a human younger than ten years old. In one embodiment, the subject is a human younger than nine years old. In one embodiment, the subject is a human younger than eight years old. In one embodiment, the subject is a human younger than seven years old. In one embodiment, the subject is a human younger than six years old. In one embodiment, the subject is a human younger than five years old. In one embodiment, the subject is a human younger than four years old.

In one specific embodiment, the subject is a human younger than three years old. In one further specific embodiment, the subject is younger than two years old. In one further specific embodiment, the subject is younger than eighteen months old. In one further specific embodiment, the subject is younger than twelve months old. In one further specific embodiment, the subject is younger than ten months old. In one further specific embodiment, the subject is younger than eight months old. In one further specific embodiment, the subject is younger than six months old. In one further specific embodiment, the subject is younger than three months old. In one further specific embodiment, the subject is a newborn.

According to a first embodiment, the subject who is treated for the keratoderma is a pre-walk human. According to a second embodiment, the subject who is treated for the keratoderma is a post-walk human. In one embodiment, a “pre-walk human” is a human child from 0 to 10 months old, from 0 to 11 months old, from 0 to 12 months old, from 0 to 13 months old, from 0 to 14 months old, from 0 to 15 months old, from 0 to 16 months old, from 0 to 17 months old or from 0 to 18 months old; depending on the development of a specific child and without being limited thereto.

In the topical treatment of keratoderma, an appropriate dosage level will generally be 0.1 to 10 mg of composition per square centimeter (cm²) of skin of the subject administrated from one to five times per day. According to one embodiment, the dosage level is about 0.1 to about 10 mg, preferably about 0.05 to about 5 mg, more preferably about 0.1 to about 2.5 mg, of composition per cm² of skin of the subject administrated from one to three times per day. According to one embodiment, the dosage level is 0.5 to 5 mg of composition per cm² of skin of the subject administrated from one to three times per day. According to one embodiment, the dosage level is about 0.5 to about 5 mg, preferably about 0.75 to about 2.5 mg, more preferably about 1.25 to about 1.75 mg, of composition per cm² of skin of the subject administrated from one to three times per day. In one embodiment, the dosage level is 1 to 3 mg of composition per cm² of skin of the subject administrated from one or two times per day. In one embodiment, the dosage level is about 1 to about 3 mg, preferably about 1.5 to about 2.5 mg, more preferably about 1.5 to about 2 mg, of composition per cm² of skin of the subject administrated from one to three times per day.

It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition and the host undergoing therapy.

This invention also relates to a fabric support comprising erlotinib in an amount ranging from 0.0001 to 5%, by weight of the total weight of the support. According to one embodiment, the fabric support comprises erlotinib in an amount ranging from about 0.0001 to about 5%, preferably from about 0.001 to about 0.5%, more preferably from about 0.01 to about 0.1%, by weight of the total weight of the support. The fabric support may be woven or non-woven. According to one embodiment, the support comprises erlotinib in an amount ranging from 0.001 to 2%, by weight of the total weight of the support. According to one embodiment, the fabric support comprises erlotinib in an amount ranging from about 0.001 to about 2%, preferably from about 0.01 to about 0.2%, more preferably from about 0.1 to about 0.2%, by weight of the total weight of the support. In one embodiment, the support comprises erlotinib in an amount ranging from 0.01 to 0.1%, by weight of the total weight of the support.

This invention also relates to a dressing or a patch comprising a support as described hereinabove. This invention also relates to a piece of clothing comprising a support as described hereinabove. According to one embodiment, the piece of clothing is a glove. In the meaning of the invention, a glove encompasses complete or partial gloves (including fingerless gloves) of any size and length (including gloves covering partially the arm). According to one embodiment, the piece of clothing is a sock. In the meaning of the invention, a sock encompasses complete or partial socks (including a fingerless socks) of any size and length (from low to high socks, including socks covering partially the leg). In one embodiment, the sock is a moisturizing gel sock, i.e., a sock further impregnated by a moisturizing gel (such as NatraCure® gel sock). According to one embodiment, the glove or sock is itself part of another clothing. This invention also relates to a spray comprising a composition for use as described hereinabove and means for providing an aerosolized mixture from this composition.

This invention also relates to the use of topical composition comprising erlotinib, as described hereinabove, in the manufacture of a medicament for the treatment of a keratoderma. This invention also relates to a method for the treatment of a keratoderma in a subject in need thereof, comprising a step of topically administrating to said subject a therapeutically effective amount of erlotinib, as described hereinabove.

### Examples

The present invention is further illustrated by the following examples.

### Example 1: Erlotinib topical compositions

### Erlotinib Suspensions

| Compound | % w/v | Compound | % w/v |
|---|---|---|---|
| erlotinib | 1 | erlotinib | 0.5 |
| methylcellulose | 2 | methylcellulose | 5 |
| ethanol | 9 | isopropanol | 2 |
| water | q.s. | water | q.s. |

| Compound | % w/v | Compound | % w/v |
|---|---|---|---|
| erlotinib | 2 | erlotinib | 1 |
| Hydroxypropylmethyl | 5 | Hydroxypropylmethyl | 0.5 |
| cellulose | | cellulose | |
| isopropanol | 1 | ethanol | 10 |
| water | q.s. | water | q.s. |

| Compound | % w/v | Compound | % w/v |
|---|---|---|---|
| erlotinib | 2 | erlotinib | 0.5 |
| carbomer | 4 | carbomer | 0.5 |
| ethanol | 3 | isopropanol | 7 |
| water | q.s. | water | q.s. |

### Erlotinib Emulsions

| Compound | % w/v |
|---|---|
| erlotinib | 0.5 |
| beta-cyclodextrin | 9 |
| hydroxypropylmethyl cellulose | 1 |
| water | q.s. |

| Compound | % w/v |
|---|---|
| erlotinib | 2 |
| Beta-cyclodextrin | 5 |
| Hydroxypropylmethyl cellulose | 3 |
| water | q.s. |

### Erlotinib Solutions

| Compound | % w/v |
|---|---|
| erlotinib | 2 |
| Transcutol ® | 15 |
| water | q.s. |

| Compound | % w/v |
|---|---|
| erlotinib | 1 |
| Transcutol ® | 10 |
| PEG 400 | 8 |
| water | q.s. |

| Compound | % w/v |
|---|---|
| erlotinib | 1 |
| Transcutol ® | 40 |
| carbomer | 2 |
| water | q.s. |

### Example 2: Devices for Topical Administration of Erlotinib

### Erlotinib Dressing:

Erlotinib-containing dressings are prepared as follows: 1 part of each of the compositions described in Example 1 (active gel) is deposited on 10 parts of a non-woven polyester support, thereby providing an erlotinib-containing support comprising from 0.05 to 0.2% w/v of erlotinib, depending on the composition. The active gel is then protected by a polyethylene film until topical application. This dressing is applied on the affected tissues of the body of a subject, e.g., a hand or a foot, in order to provide topical application of the composition to the skin of the subject. Transdermal release of erlotinib allows continuous treatment of the affected tissues by erlotinib.

### Erlotinib Sock:

Erlotinib-containing socks are prepared as follows: 20 parts of a woven fabric support are impregnated with 1 part of each of the compositions described in Example 1, thereby providing an erlotinib-containing support comprising from 0.025 to 0.1% w/v of erlotinib, depending on the composition. The support is then sewed on the internal part of a commercial sock. This sock may be worn by a subject in order to provide topical application of the composition to the foot of the subject. Transdermal release of erlotinib allows continuous treatment of the skin of the foot by erlotinib.

### Example 3: Topical Use of Erlotinib for Treating a Keratoderma

### Materials and Methods

Topical Application of the Composition:

Topical compositions are preferably applied to cleaned skin after bathing or showering. A cream or ointment is applied either with fingers of a gloved hand (with a plastic or vinyl or latex glove), with a spatula or with a non-sterile cotton swab. Ointment have an occlusive effect. Other topical formulations are applied according to directions for use and medical indications. Topical compositions should normally cover only the lesions. The penetration of creams and ointments may be improved by a light massage. The patient is installed comfortably. It is waited a few minutes before dressing her or him after the topical application. The effects of the treatment are observed locally from day to day. The patient is interrogated and listened on what he feels.

### Criteria for Evaluation in PPK

### Kinetics:

Systemic absorption is evaluated through measurement of serum erlotinib.

### Clinical Evaluation

Clinical evaluation is carried out by the medical staff such as for example a physician or an investigor. Various means known in art can be used, such as for standardized photographs.

Any one or all of the following criteria may be used:
assessment of decreased hyperkeratosis,
Gross Motor Function Measure (EMFG) is an assessment tool designed and evaluated to measure changes in gross motor function over time or with intervention in children with cerebral palsy,
pruritus score,
number of steps taken as assessed by activity monitor,
footprint to assess deformation and his evolution,
necessity of the use of wellchair and/or crutches and evolution thereof,
determination of the difference between treated and untreated foot in change of pressure (measured by manometry) to reach unbearable pain (VAS-Pain Scale),
assessment of local tolerability at the application sites (VAS-pain Scale)
determination of safety/toxicity of erlotinib by recording of reported adverse events (AE), and
measurement of consumption of analgesics, corticoids and other treatments.

### Quality of Life (QoL) Assessment

Any one or all of the following quality of life scales are used:
PGA: Patient Global Assessment of Activities Scale (patient's global assessment of disease activity (PTGL): disease activity and global health.
CGI-I: Clinician Global Impression Improvement Scale: a 7-point scale that requires the clinician to assess how much the patient's illness has improved or worsened relative to a baseline state at the beginning of the intervention. and rated as: very much improved/much improved/minimally improved/no change/minimally worse/much worse/very much worse.
VAS-Pain: Visual Analog Scale for Pain questionnaire to assess pain in feet intensity, the scale is most commonly anchored by “no pain” (score of 0) and “pain as bad as it could be” or “worst imaginable pain” (score of 100 [100-mm scale]).
Marjorie Gordon functional health patterns (11 items): health perception and health management, nutrition and metabolism, elimination, activity and exercise, cognition and perception, sleep and rest, self-perception and self-concept, roles and relationships, sexuality and reproduction, coping and stress tolerance, values and belief.
HDRS (Ham-D), Hamilton Depressive Rating Scale: the most widely used clinician-administered depression assessment scale. The original version contains 17 items (HDRS17) pertaining to symptoms of depression experienced over the past week
Tanner Scale or Sexual Maturity Rating (SMR) is a scale of physical development in children, adolescents and adults. The scale defines physical measurements of development based on external primary and secondary sex characteristics, such as the size of the breasts, genitals, testicular volume and development of pubic hair.
Self-administered questionnaires (six):
   BoPPK: Burden of PalmoPlantar Keratoderma on 56 items in 24 questions and 4 factors are highlighted: relationships with others, work and life activities, self-image, psychological impact.
   DLQI: Dermatology Life Quality Index in 10 questions about: skin condition, embarrassed/self-conscious, interference with activities, clothes, social/leisure activities, sport, working/studying, problems with partner/friends/relatives, sexual difficulties, home messy/taking up time.
   PSS: Perceived Stress Scale in 10 questions, in the last month, how often have you felt that: upset because of something that happened unexpectedly? unable to control the important things in your life? nervous and “stressed”? confident about your ability to handle your personal problems? that things were going your way? that you could not cope with all the things that you had to do? you have been able to control irritations in your life? that you were on top of things? you have been angered because of things that were outside of your control? difficulties were piling up so high that you could not overcome them?
   SF12 (Short Form 12): 12 questions about the past 4 weeks: health condition? health limiting moderate activities? (moving a table, pushing a vacuum cleaner, bowling, or playing golf), health limiting climbing several flights of stairs? physical health on regular activities accomplished less than you would like? physical health on regular activities limited in the kind of work or other activities? emotional problems results: accomplished less than you would like? emotional problems results: work or other activities not done as carefully as usual? pain interfere with your normal work? (including both work outside the home and housework), feel calm and peaceful? have a lot of energy? felt downhearted and blue? physical or emotional problems interfered with social activities (like visiting with friends, relatives, etc.)?
   Skindex: on 61 items on 8 scales: cognitive effects, social effects, depression, fear, embarrassment, anger, physical discomfort, and physical limitation.
   Zarit Caregiver Burden Scale: 22-item questionnaire assesses the “experience of burden.”: do you feel that your relative asks for more help than he or she needs? do you feel that because of the time you spend with your relative you do not have enough time for yourself? do you feel stressed between caring for your relative and trying to meet other responsibilities for your family or work? do you feel embarrassed over your relative's behaviour? Do you feel angry when you are around your relative? Do you feel that your relative currently affects your relationship with other family members or friends in a negative way? are you afraid about what the future holds for your relative? do you feel your relative is dependent on you? Do you feel strained when you are around your relative? Do you feel your health has suffered because of your involvement with your relative? do you feel that you do not have as much privacy as you would like because of your relative? do you feel that your social life has suffered because you are caring for your relative? do you feel uncomfortable about having friends over, because of your relative? do you feel that your relative seems to expect you to take care of him or her, as if you were the only one he or she could depend on? Do you feel that you do not have enough money to care for your relative, in addition to the rest of your expenses? do you feel that you will be unable to take care of your relative much longer ? do you feel you have lost control of your life since your relative's illness ? do you wish you could just leave the care of your relative to someone else? do you feel uncertain about what to do about your relative? Do you feel you should be doing more for your relative? do you feel you could do a better job in caring for your relative? overall, how burdened do you feel in caring for your relative?

### Results:

Success of the topical treatment of the keratoderma is evaluated using kinetic evaluation, clinical evaluation and/or quality of life (QoL) evaluation.

### Example 4: Topical Use of Erlotinib in the Topical Treatment of a Keratoderma in Children

### Materials and Methods

Erlotinib Administration:

The patients are children showing symptoms of a plantar keratoderma. The children are typically aged from eleven to fifteen years. The children typically have a high level of pain as measured by methods known in the art (e.g., VAS-pain Scale). The topical application of the erlotinib composition takes place once a day for three months on the palms of the hands and/or the soles of the feet of the patient. Topical compositions should normally cover only the lesions. The erlotinib composition is either a cream or a gel. The erlotinib composition is preferably applied to cleaned skin after bathing or showering. The erlotinib composition is applied either with fingers of a gloved hand (with a plastic or vinyl or latex glove), with a spatula or with a non-sterile cotton swab, without massaging nor rubbing. Then, the skin is covered with a dressing (bandage).

### Criteria for Evaluation in PPK

Quality of life (QoL) assessment: A pain assessment is performed through recognition of indications of pain on the face of the child. This recognition can be done by the parents and/or a healthcare professional, optionally assisted by medical device comprising a recording device, a facial recognition software and a computer.

### Clinical Evaluation:

The possible appearance of hyperkeratotic lesions is also monitored. When relevant, other criteria for evaluation in PPK as described in Example 3 hereinabove are also used.

### Results:

Success of the topical treatment of the keratoderma is evaluated using kinetic evaluation, clinical evaluation and/or quality of life (QoL) evaluation.

### Example 5: Topical Use of Erlotinib in a Sequential Oral and Topical Treatment of a Keratoderma in Children

### Materials and Methods

### Erlotinib Administration:

The patients are children presenting plantar lesions and/or keratoderma and Olmsted or Pachyonychia congenita symptoms. The children are typically aged from eleven to fifteen years. The children typically have a high level of pain as measured by methods known in the art (e.g., VAS-pain Scale). The patients are treated through a sequential treatment: (1) first oral erlotinib treatment according to directions for use and medical indications, then (2) erlotinib topical treatment as described herein, when the intensity of the lesions and/or plantar keratoses has diminished. The topical application of the erlotinib composition takes place once a day for three months on the palms of the hands and/or the soles of the feet of the patient. Topical compositions should normally cover only the lesions. The erlotinib composition is either a cream or a gel. The erlotinib composition is preferably applied to cleaned skin after bathing or showering. The erlotinib composition is applied either with fingers of a gloved hand (with a plastic or vinyl or latex glove), with a spatula or with a non-sterile cotton swab, without massaging nor rubbing. Then, the skin is covered with a dressing (bandage).

### Criteria for Evaluation in PPK:

The criteria for evaluation in PPK are as described in Example 3 hereinabove.

### Results:

Success of the treatment of the keratoderma is evaluated using kinetic evaluation, clinical evaluation and/or quality of life (QoL) evaluation.

1-15. (canceled)

16. A method of treatment of a keratoderma in a subject in need thereof, comprising a step of topical administrating to said subject of a therapeutically effective amount of a topical composition;
wherein said composition comprises:
erlotinib in an amount ranging from about 0.01 to about 10% by weight of the total weight of the composition and
at least one pharmaceutically acceptable excipient; and
wherein said subject is a human younger than fifteen years old.

17. The method according to claim 16, wherein said keratoderma is a palmoplantar keratoderma.

18. The method according to claim 17, wherein said palmoplantar keratoderma is an inherited palmoplantar keratoderma.

19. The method according to claim 18, wherein said inherited palmoplantar keratoderma is selected from the group consisting of inherited diffuse palmoplantar keratoderma and inherited focal palmoplantar keratoderma.

20. The method according to claim 19, wherein said inherited palmoplantar keratoderma is selected from the group consisting of Erythrokeratodermia variabilis, palmoplantar keratoderma of Sybert, Olmsted syndrome, and Naegeli Franceschetti Jadassohn syndrome; Papillon-Lefevre syndrome, Pachyonychia congenita type I, Pachyonychia congenita type II, focal palmoplantar keratoderma with oral mucosal hyperkeratosis, and Camisa disease.

21. The method according to claim 20, wherein said palmoplantar keratoderma is selected from the group consisting of Olmsted syndrome, Pachyonychia congenita type I, and Pachyonychia congenita type II.

22. The method according to claim 16, wherein said composition comprises erlotinib in an amount ranging from about 0.1 to about 5% by weight of the total weight of the composition.

23. The method according to claim 16, wherein said pharmaceutically acceptable excipient comprises a penetration enhancer selected from the group consisting of an alcohol, a polyoxyethylene sorbitan monooleate, a polyoxyethylene (C10 C14) alkyl ether, a propylene glycol (C6 C10) alkyl ester, a polyethylene glycol (C6 C10) alkyl ester, a polyethylene glycol, a cyclodextrin, and a mixture thereof.

24. The method according to claim 23, wherein said penetration enhancer is selected from the group consisting of ethanol, isopropanol, 2 (2 ethoxyethoxy)ethanol, polysorbate 20, polysorbate 80, polyoxyethylene (4) lauryl ether, propylene glycol monocaprylate, polyethylene glycol caprylic ester, PEG 400, βcyclodextrin, and a mixture thereof.

25. The method according to claim 16, wherein said composition is a cream, a liniment, a gel, a lotion, an ointment, a foam, a solution, a suspension, an emulsion, a paste, an aerosolized mixture, or a powder.

26. The method according to claim 16, wherein said composition is administered topically to a lesion of the skin of said subject.

27. The method according to claim 16, wherein said composition is administered topically to a hand and/or a foot of said subject.

28. The method according to claim 16, wherein said method further comprises a step of oral administration of erlotinib to said subject, in addition of the topical administration of said composition to said subject.

29. The method according to claim 28, wherein said method comprises oral administration of erlotinib to said subject before and/or after the topical administration of said composition to said subject.

30. The method according to claim 16, wherein said subject is a human younger than thirteen years old.

31. The method according to claim 30, wherein said subject is a human younger than ten years old.

32. The method according to claim 16, wherein said subject is a human younger than five years old.

33. A woven or non-woven fabric support comprising erlotinib in an amount ranging from about 0.001 to about 2% by weight of the total weight of the support.

34. The support according to claim 33, wherein said support comprises erlotinib in an amount ranging from about 0.01 to about 0.1%, by weight of the total weight of the support.

35. A dressing, patch, glove or sock comprising the support according to claim 33.
